(19) United States
(12) Patent Application Publication
(10) Pub. No.: US 2023/0335291 A1
(43) Pub. Date: Oct. 19, 2023

(54) HEALTH MONITORING SYSTEMS

(71) Applicant: Adritha Technologies LLC, Novi, MI (US)

(72) Inventors: Venkata Prasad ATLURI, Novi, MI (US); Virith KAZA, Novi, MI (US)

(21) Appl. No.: 18/136,201

(22) Filed: Apr. 18, 2023

Related U.S. Application Data
(60) Provisional application No. 63/332,045, filed on Apr. 18, 2022.

(51) Int. Cl. G16H 50/30; A61B 5/0205; A61B 5/00; G16H 15/00; G16H 40/67

(52) U.S. Cl. CPC G16H 50/30; A61B 5/0205; A61B 5/0022; G16H 15/00; G16H 40/67

(57) ABSTRACT

A system includes a transceiver configured to communicate health data to a remote server, one or more processors in communication with the transceiver wherein the one or more processors are programmed to receive an input at a user interface of the system, activate a switch to activate one or more sensors in communication with the system, wherein the one or more sensors are configured to collect one or more of an image of the user, temperature data, blood pressure data, heart rate data, glucose data, or echocardiogram data, send data via the wireless transceiver, to the remote server to identify one or more health conditions associated with the user, and in response to the identifying the one or more health conditions exceeding a threshold, output a report indicative of one or more health assessments indicating a condition or an illness associated with the user.

### CROSS-REFERENCE TO RELATED APPLICATIONS

100911 This application claims the benefit and priority of U.S. Application Ser. No. 63/332,045 filed Apr. 18, 2022, the disclosure of which is hereby incorporated in its entirety by reference herein.

### TECHNICAL FIELD

The present disclosure relates to health monitoring systems, including those that have the ability to read data from various patients or users and process the data on-board or off-board.

### BACKGROUND

The increase in the rate of population of the elders and the increased life expectancy has created numerous challenges in the healthcare industry. Common day to day human activities are affected for those elders who are suffering from health issues like cardio vascular health problems, arthritis, mobility impairment, diabetes, dementia, Alzheimer's, Parkinson's. The elders who do not get health support may have health risks, such as depression or other disease. It may be beneficial to allow more frequent tracking of vital signals and utilizing such signals for periodic monitoring.

### SUMMARY

According to a first embodiment, a health monitoring system utilized for a user includes an enclosure configured to hold one or more components of the health monitoring system. The enclosure includes a wireless transceiver configured to communicate health data to a remote server, a camera configured to capture an image associated with a user, a temperature sensor configured to capture temperature data indicative of a body temperature indicative of the user, a blood pressure sensor configured to capture blood pressure data indicative of a blood pressure of the user, a heart rate sensor configured to capture heart rate data indicative of a heart rate of the user, a glucose sensor configured to capture glucose data indicative of a glucose level of the user, an electrocardiogram (ECG) configured to record an electrical signal associated with a heart of a user to capture ECG data and one or more processors in communication with the wireless transceiver, camera, temperature sensor, blood pressure sensor, heart rate sensor, glucose sensor, and ECG, wherein the processors are collectively further programmed to receive the images, the temperature data, the blood pressure data, the heart rate data, and the glucose data, the ECG data, send, via the wireless transceiver, to the remote server the images, the temperature data, the blood pressure data, the heart rate data, and the glucose data, the ECG data, utilizing a machine learning model and the image, the temperature data, the blood pressure data, the heart rate data, and the glucose data, the ECG data , identify one or more health conditions associated with the user, and in response to the identifying the one or more health conditions exceeding a health threshold, output a health report indicative of one or more health assessments indicating a healthy condition or an illness associated with the user.

According to a second embodiment, a health monitoring system utilized for a user includes a wireless transceiver configured to communicate health data to a remote server, one or more processors in communication with the wireless transceiver wherein the one or more processors are collectively further programmed to upon receiving an input at a user interface of the health monitoring system, activate a switch to activate one or more sensors in communication with the health monitoring system, wherein the one or more sensors are configured to collect one or more of an image of the user, temperature data, blood pressure data, heart rate data, glucose data, or echocardiogram data, send, via the wireless transceiver, to the remote server the images, the temperature data, the blood pressure data, the heart rate data, and the glucose data, the ECG data, utilizing a machine learning model and one or more of the image, the temperature data, the blood pressure data, the heart rate data, and the glucose data, the ECG data, identify one or more health conditions associated with the user, and in response to the identifying the one or more health conditions exceeding a health threshold, output a health report indicative of one or more health assessments indicating a healthy condition or an illness associated with the user.

According to a third embodiment, a method of monitoring a health of a user utilizing a personal health monitoring system, wherein the method includes utilizing a camera, receiving an image associated with a user, utilizing a temperature sensor, receiving temperature data indicative of a body temperature indicative of the user, utilizing a blood pressure monitor, receiving blood pressure data indicative of a blood pressure of the user utilizing a blood pressure monitoring, utilizing a heart rate sensor, receiving heart rate data indicative of a heart rate of the user, utilizing a glucose monitor, receiving glucose data indicative of a glucose level of the user, utilizing an electrocardiogram (ECG), receiving an electrical signal associated with a heart of a user to capture ECG data, receiving, via one or more processor, the images, the temperature data, the blood pressure data, the heart rate data, and the glucose data, the ECG data, sending, via the wireless transceiver, to the remote server the images, the temperature data, the blood pressure data, the heart rate data, and the glucose data, the ECG data, utilizing a machine learning model and the image, the temperature data, the blood pressure data, the heart rate data, and the glucose data, the ECG data, identifying one or more health conditions associated with the user, and in response to the identifying the one or more health conditions exceeding a health threshold, outputting a health report indicative of one or more health assessments indicating a healthy condition or an illness associated with the user.

### BRIEF DESCRIPTION OF THE DRAWINGS

FIG. 1 illustrates an overview of a health monitoring system according to an embodiment.

FIG. 2 illustrates a diagram of a health monitoring system according to an embodiment.

FIG. 3 illustrates a diagram of a system with remote services, such as a connected care service.

FIG. 4 illustrates user interface screens associated with the personalized health monitoring systems.

FIG. 5 illustrates a flowchart associated with the actions of the personalized health monitoring system according to one embodiment.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Aspects of the present disclosure relate to health monitoring systems and more specifically, a personalized health monitoring and alerting system. As shown in the figures, a health monitoring system according to one or more embodiments of the present disclosure may include a user-side monitoring device and a remote server (e.g. a cloud) in communication with the monitoring device.

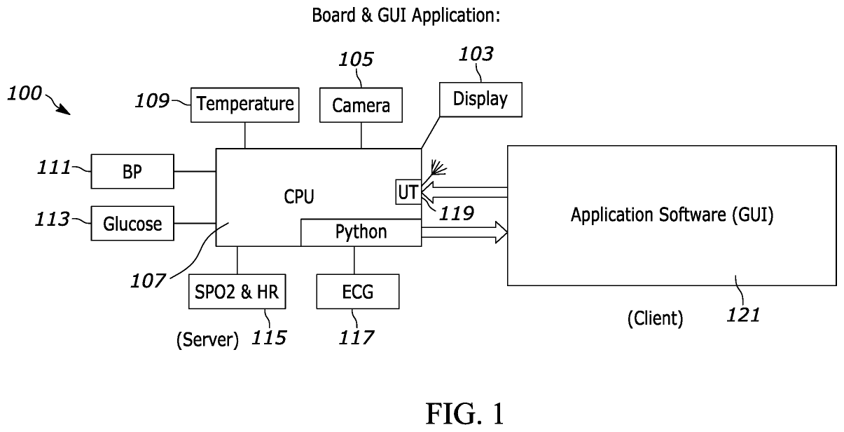

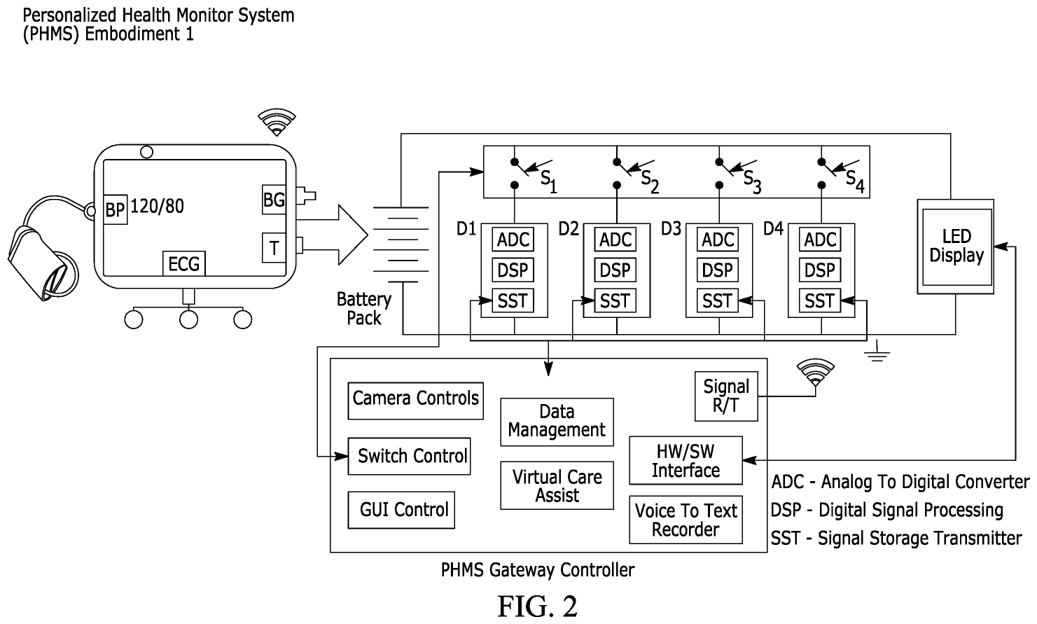

FIG. 1 illustrates an overview of a first embodiment of a health monitoring system. FIG. 2 illustrates a diagram of a health monitoring system according to an embodiment. Referring to the figures, the monitoring device 100 may include at least one monitoring unit configured to measure electronic health signals (e.g. vital signs) of a user. Some examples of the electronic health signals may include a heart rate, a blood pressure (BP), a blood oxygen saturation (SpO2) level, a blood glucose level, a body temperature, skin or facial conditions, and electrocardiogram (ECG) data of the user. In some embodiments, the monitoring device may be wearable by the user. The at least one monitoring unit may be one or more sensors (e.g. biosensors).

In some embodiments, the monitoring device may include one or more battery pack 180 configured to provide electric power thereto. In some other embodiments, the monitoring device may include a charging port for charging thereof. In yet some other embodiments, the monitoring device may be configured to be charged wirelessly.

The monitoring device may further include at least one switch system 160 configured to control the operation of the at least one monitoring unit. For example, the at least one switch may be configured to turn on or turn off one or more of the at least one monitoring unit. The at least one switch may be operably controlled by a switch control unit 169 of the monitoring device as described hereafter. In some embodiments, one switch may control the operation of one monitoring unit of the monitoring device. In some other embodiments, one switch may simultaneously control the operation of more than one monitoring unit of the monitoring device. A series of one or more switches may be connected to the individuals in the PHMS and that are controlled by the gateway controller. The controller may be controlled these switches based on the input selection from the users. These switches (on/off states) may provide initialization message to devices to wake up via an algorithm store on memory at the gateway controller.

The monitoring device may also include at least one circuit electronically coupled to or integrated with the at least one switch 160. The at least one circuit may further be operably connected to the switch control unit of the monitoring device. Each circuit 165a, 165b, 165c, 165d, may include an analog to digital converter (ADC), a digital signal processor (DSP), and a signal storage and transmitter (SST). In some embodiments, one circuit may be electronically coupled to or integrated with one switch. In some other embodiments, one circuit may be electronically coupled to or integrated with more than one switch. The monitoring device 100 may include a processor 101 to execute any flow charts, algorithms, and/or processes.

The monitoring device 100 may include a processor 101 that includes an application programming interface to communicate with one or more sensors. In one scenario, the one or more sensors described herein as related to the health readings. The system may utilize various drivers that include a programming interface to control and manage interfaces linked to the hardware of the sensors. The sensors may be in communication with the processor 107 via a hard-wire connection or wireless connection (e.g. Wi-Fi, Bluetooth, or any wireless communication protocol).

The processor 107 may store various APIs or drivers related to a camera 105, a temperature sensor 107, a blood pressure monitor 111, a glucose monitor 113, a heart rate monitor 115, and an echocardiogram (ECG) monitor 117. The driver may be utilized to allow for any auxiliary device to handshake between the various devices. In one example, TCP/IP may be utilized to manage the exchange of data between the server (e.g. gateway controller and sensors) and the client (e.g. interface). For example, the handshaking software may allow the GUI to display various data and readings from the various sensors.

The monitoring device may further include a display unit 103 configured to allow the user to control the operation of the monitoring device and to interact with the monitoring device. In some embodiments, the display unit 103 may include a touch screen, through which the user may control the operation of the monitoring device and interact with the monitoring device. In one embodiment, the display unit is a 7″ LCD screen. In some other embodiments, the display unit 103 may be electrically coupled to an external input device, through which the user may control the operation of the monitoring device and interact with the monitoring device. Some examples of the external input device may be a mouse, a keyboard, or a voice-based device. For example, the user may track the user's health conditions through the display unit. The user may input, via the display unit, instructions to the monitoring device to begin a health measurement event. The user may also view the progress of the health measurement event via the display unit. The user may further review, via the display unit, data related to the health measurement event at the completion of the health measurement event. The user may also select, through the display unit, to save, delete or copy the data related to the health measurement event. The user may further select, through the display unit, to transmit the data related to the health measurement event to the remote server of the health monitoring system for storage or further analysis. The user may also view a user's health profile through the display unit. The user may provide voice instructions or commands to the display unit. The display unit may also prompt or display alerts (e.g. health alerts) to the user. In some embodiments, the display unit may be a light-emitting diode (LED) display or a liquid-crystal display (LCD) screen.

The monitoring device may also include camera 105 or a video capturing unit 105 configured to capture a video and/or image of the user. For example, the video capturing unit 105 may capture a video and/or an audio of the user while the health measurement event is in progress. The video and/or the audio of the user may indicate the user's reactions, including facial or body expressions, to the health conditions of the user, which may be utilized by the health monitoring system when analyzing the user's health conditions. The video capturing unit 105 may capture a video and/or image of the user to recognize skin and/or facial conditions of the user (e.g. to recognize redness, pale, fatigue, rashes, or the like). The video capturing unit 105 may also receive questions, answers, instructions, and/or commands from the user regarding the user's health conditions. In some embodiments, the video capturing unit 105 may be a camera (e.g. an artificial intelligence camera), an optical sensor, a vision recognizer, or the like.

The monitoring device may further include a gateway controller 107 configured to control the operation of the monitoring device. The gateway controller 107 may be electronically coupled to the monitoring device or integrated with the monitoring device. As shown in the figures, the gateway controller may include a first control unit configured to control the operation of the at least one switch of the monitoring device. The first control unit may be a switch control unit. As discussed herein, the first control unit may be operably connected to the at least circuit of the monitoring device.

The gateway controller 107 may also include a second control unit configured to control the operation of the video capturing unit of the monitoring device. The second control unit may be a camera control unit 167. The video capturing unit 105 may be a camera (e.g. an artificial intelligence (AI)-based camera), an optical sensor, a vision recognizer, or the like.

The gateway controller 107 may further include a third control unit configured to control the operation and functions of the display unit of the monitoring device. The third control unit may be a graphical user interface (GUI) control unit 121 or other input interface.

The gateway controller 107 may also include a data management unit 151 configured to store and/or process data in relation to the user's health conditions. Such data may include, but not limited to, data related to the user's health measurement events, the user's health profiles, and the user's health alerts. In some embodiments, the data management unit may be configured to generate and/or manage (e.g. update) a health profile of the user based on the user's electronic health signals.

The gateway controller 107 may further include a virtual care assisting unit configured to provide real-time assistance to the user. For example, the virtual care assisting unit may be configured to provide instructions to the user on how to operate the monitoring device and/or on how to communicate with the remote server. The virtual care assisting unit may also be configured to provide a chat function such that the user may conduct live conversations with a health expert (e.g. a health care provider or practitioner) via the display unit of the monitoring device. The virtual care assisting unit may further be configured to include preloaded questionnaires to help the user self-evaluate the user's health conditions. The virtual care assisting unit may also be configured to prompt a health alert to the user and to assist the undertaking of a virtual meeting between the user and the health expert when it is needed.

The gateway controller 107 may also include a data transceiver configured to communicate data between the monitoring device and the remote server. For example, the data transceiver 119 may transmit data regarding the user's health conditions from the monitoring device to the remote server. Such data may include, but not limited to, data related to the user's health measurement events and the user's health profiles. The data transceiver may also receive, among others, health condition analysis reports, health alerts, health instructions or tips, or the like from the remote server. In some embodiments, the data transceiver may communicate wirelessly between the monitoring device and the remote server. In some other embodiments, the monitoring device may be electronically connected to the remote server using cables or wires.

The gateway controller 107 may further include a hardware and software interface configured to control the operation of the monitoring device. The hardware and software interface may be configured to control the operation of the first, second, and/or third control unit as described herein.

The gateway controller 107 may also include a voice recorder 181 configured to receive voice instructions or commands from the user. The voice recorder may also be configured to convert the voice instructions or commands into text instructions or commands, which allows the user to communicate with the monitoring device by speaking.

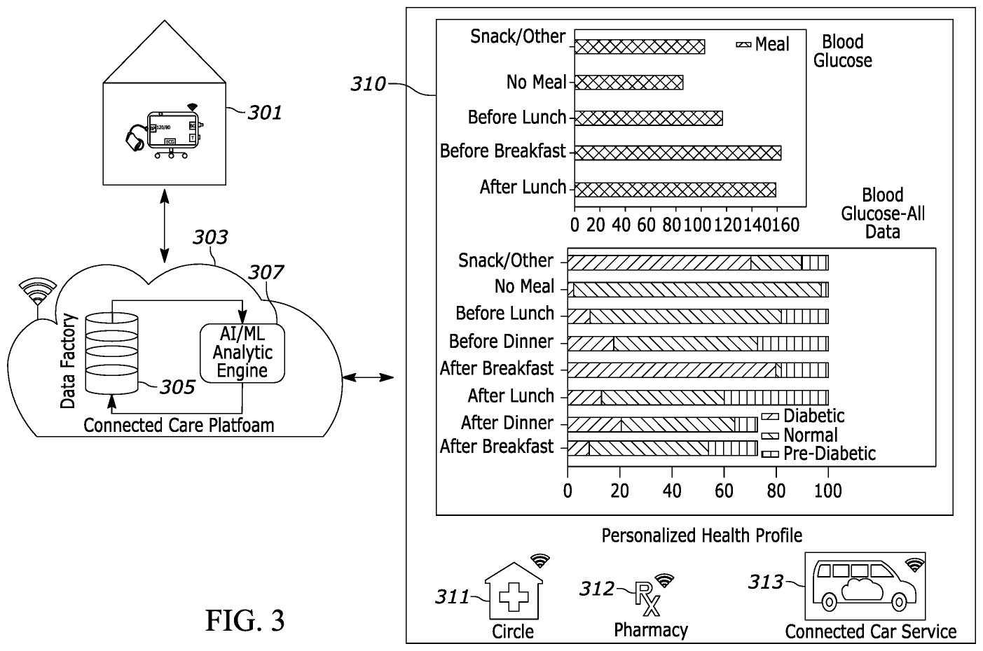

FIG. 3 illustrates an overview of a connected system with the personal health monitoring system. Continuing referring to the figures, the health monitoring system 301 may further include a remote server 303 (e.g. a cloud) in communication with the monitoring device. The remote server 303 may include a data factory 305 including a health-related database. The database may include, but not limited to, the user's past health profiles, the user's past health condition analysis reports, and the user's past health alerts. The database may further include other data related to common health condition patterns, concerns and/or symptoms.

The remote server 303 may further include an analytics engine 307 configured to analyze the received data to ultimately determine the user's health conditions. The received data may include, but not limited to, data related to the user's health measurement events and the user's health profiles. The analytics engine may include one or more algorithms configured to monitor, identify, and/or predict a health issue or concern based on the received data. The thresholds for identifying any health issues or concerns may be preset by health experts. The thresholds may also be based on the data stored in the health-related database of the data factory. In addition, the thresholds may be determined by a pattern recognition algorithm implemented in the analytics engine.

For example, the pattern recognition algorithm may be configured to identify abnormal health condition patterns and/or symptoms based on the received data. These abnormal health condition patterns and/or symptoms may be taken by the analytics engine as indications of potential health issues or concerns of the user. Thereupon, the remote server may generate a health alert and may then transmit the health alert to the monitoring device.

The health alert may be presented to the user through the display unit of the monitoring device. The health alert may also be presented to the user through voice signals generated by the monitoring device. The health alert may further be presented to the user via text messages and/or email reports or notifications. The health alert may also be transmitted to health experts, especially when further diagnosis is necessary. The thresholds for alerting abnormal health condition patterns and/or symptoms of the user may be based on, but not limited to, health signals (e.g. short-time horizon) of the user, the state of health (e.g. long-time horizon) of the user, and recommended ranges of values set by health experts. The thresholds may be set for each individual user of the system. The thresholds may be continuously updated based on incoming health signals or reports related to the user, and/or any estimated or computed state of health of the user.

In some embodiments, the analytics engine may be configured to update the received health profile 310 of the user and to store the updated health profile in the data factory. In some other embodiments, the analytics engine may be configured to generate a new health profile for the user indicating the identified health issue or concern and to store the new health profile in the data factory.

The analytics engine may utilize machine learning techniques to map health data from the user to identify patterns for certain issues or symptoms. By using the machine learning techniques, the analytics engine may determine the state of health of the user and report the state of health to the user. The state of health of the user may be an index or a numerical value representing a health profile of the user. Some data used by the analytics engine may include real-time health signals of the user, a facial expression of the user (e.g. captured by an AI-based camera), images or reports uploaded by the user to the system, responses of the user to questionnaires, historical health signals of the user, and others.

The remote server may also include an appointment scheduler configured to schedule a virtual clinic 311 or virtual meeting 311 for the user with a health expert based on the analysis of the user's health conditions. The virtual meeting 311 may be scheduled based on the availability of both the user and the health expert. The remote server may be configured to generate an invite (e.g. an access link) to the scheduled virtual meeting 311 and to send the invite both to the monitoring device and to the health expert. Both the user and the health expert may thus join the scheduled virtual meeting using the invite to discuss the user's health conditions. In some embodiments, before the virtual meeting is scheduled, the remote server may be configured to send an inquiry to the monitoring device to ask for the user's confirmation of making the appointment with the health expert. In some other embodiments, after the virtual meeting is scheduled, the user may select, through the display unit of the monitoring device, to cancel, postpone or reschedule the virtual meeting.

In some embodiments, the remote server may be configured to send prescriptions to pharmacy stores 312. The prescriptions may be prescribed by the health expert after the health expert discusses with the user about the user's health conditions at the virtual meeting. In some other embodiments, the remote server may be configured to communicate with a connected care service 313 to request a dispatch of a health expert, a health caregiver, an ambulance or the like to the user to, for example, administer shots or medications to the user.

A method for monitoring health conditions of a user is described. The method may monitor the health conditions of the user using the health monitoring system as described herein. The method may include measuring electronic health signals (e.g. vital signs) of the user. The method may measure the electronic health signals of the user using the monitoring device of the health monitoring system as described herein. The monitoring device may include at least one monitoring unit configured to monitor electronic health signals including, but not limited to, a heart rate, a blood pressure (BP), a blood oxygen saturation (SpO2) level, a blood glucose level, a body temperature, skin or facial conditions, and electrocardiogram (ECG) data of the user. In some embodiments, the monitoring device may be wearable by the user. The at least one monitoring unit may be sensors (e.g. biosensors).

The method may further include generating a health profile of the user based on the electronic health signals of the user measured by the monitoring device. The method may also include storing the health profile and the measured electronic health signals of the user in the monitoring device of the health monitoring system (e.g. in the data management unit of the monitoring device as described herein).

The method may further include transmitting data regarding the user's health conditions from the monitoring device to a remote server (e.g. a cloud). The remote server is part of the health monitoring system as described herein and is in communication with the monitoring device. The data may include, but not limited to, data related to the user's health measurement events and the user's health profiles. The remote server may include a data factory including a health-related database. The database may include, but not limited to, the user's past health profiles, the user's past health condition analysis reports, and the user's past health alerts. The database may further include other data related to common health condition patterns, concerns and/or symptoms.

The method may also include analyzing the received data to ultimately determine the user's health conditions. The received data may include, but not limited to, data related to the user's health measurement events and the user's health profiles. The method may analyze the received data using an analytics engine of the remote server. The analytics engine may include one or more algorithms configured to monitor, identify, and/or predict a health issue or concern based on the received data. The thresholds for identifying any health issues or concerns may be preset by health experts, and may also be based on the data stored in the health-related database of the data factory.

The method may further include identifying abnormal health condition patterns and/or symptoms based on the received data. The method may identify the abnormal health condition patterns and/or symptoms using a pattern recognition algorithm of the analytics engine. These abnormal health condition patterns and/or symptoms may be taken by the analytics engine as indications of potential health issues or concerns of the user.

The method may also include generating a health alert and transmitting the health alert to the monitoring device of the health monitoring system. The method may further include transmitting the health alert to health experts.

The method may also include updating the received health profile of the user and to store the updated health profile in the data factory. The method may further include generating a new health profile for the user indicating the identified health issue or concern and storing the new health profile in the data factory.

The method may also include scheduling a virtual meeting for the user with a health expert based on the analysis of the user's health conditions. The method may schedule the virtual meeting through an appointment scheduler of the remote server. The virtual meeting may be scheduled based on the availability of both the user and the health expert.

The method may further include generating an invite (e.g. an access link) to the scheduled virtual meeting and sending the invite both to the monitoring device and to the health expert. Both the user and the health expert may thus join the scheduled virtual meeting using the invite to discuss the user's health conditions.

The method may also include sending prescriptions to pharmacy stores. The prescriptions may be prescribed by the health expert after the health expert discusses with the user about the user's health conditions at the virtual meeting. The method may further include communicating with a connected care service to request a dispatch of a health expert, a health caregiver, an ambulance or the like to the user to, for example, administer shots or medications to the user.

The method may include creating and training a personalized health index model based on an individual user. The health index (HI) model may be developed by utilizing the different data that is read from the various sensors and applying it to the model. The HI model may output a score that identifies a particular healthiness of a user. The score may factor all of the data that is utilized in factoring an overall index of the user. For example, if all readings from the various sensors are poor, the user may receive a low overall health index score. If all the readings of the sensor are good, the user may receive a higher overall health index score.

The HI model may be personalized to a particular user of the system. The HI model may update and be trained for that individual user based on age, gender, height, weight, demographics, etc. For example, sensor readings and data that is utilized for a young person may not be useful for an older person to develop a score. Furthermore, gender may play a different role in the various readings. Once a user is logged in, the system can correlate a particular HI model from the user to utilize or train. For example, a The HI model may include an initialization phase that the system takes readings from the user. The system may update various thresholds to identify a particular reading or score based on the artificial intelligence model.

The HI model may be trained and updated at either a remote server or on the health device itself. In some embodiments, the system may be a hybrid approach that does both. The HI model may output a health report that includes an overall health score that is modeled and determined utilizing the HI model. The health repot may include an image of the user, the health score, and various readings associated with the sensors (e.g. blood pressure reading, body temperature reading, heart rate reading, blood sugar level, and ECG reading. The health report may also include a listing of various profile information of the user, such as age, gender, height, weight, etc.

The blood glucose monitor may factor in a time of day or timing of a feeding/meal when evaluating the blood glucose of the user. For example, the system may monitor blood glucose data at a snacking period, in a period of no eating (e.g. a fasting period), before/after lunch, before/after breakfast, or before/after dinner.

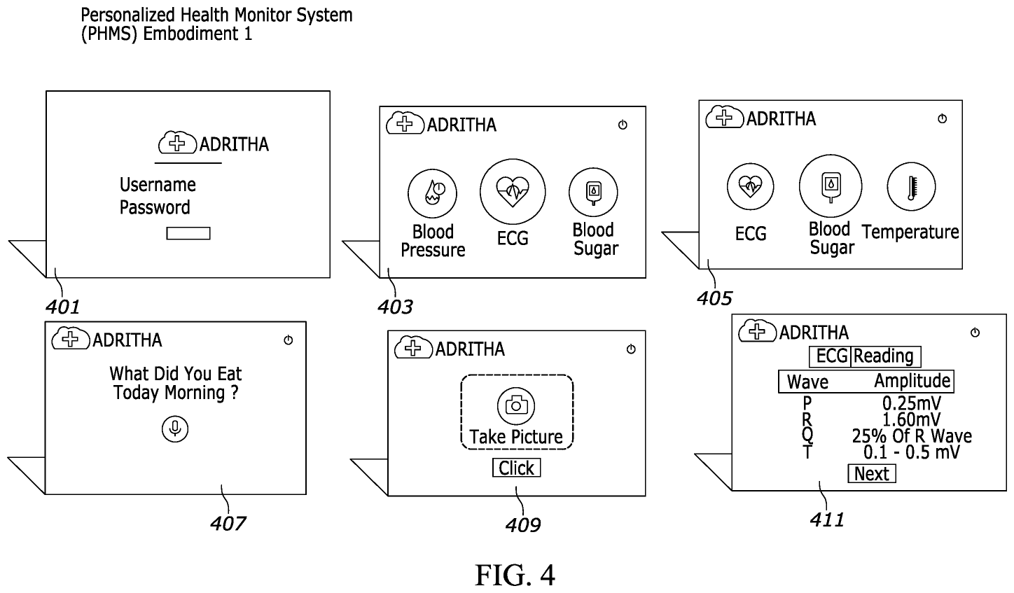

FIG. 4 illustrates various screens for the graphical user interface (GUI). The GUI may also be referred to as a human machine interface (HMI). Login screen 401 illustrates a welcome screen that requires a user to login for security and individualization purposes. The user may enter their user name and password. The two fields are verified to make sure they match to gain access to the individual user profile that includes personalized health readings or statistics, as well as a HI model. The login screen 401 may allow for multiple users to utilize the same device and then track readings for those individual profiles.

ECG screen 403 may illustrate a symbol or icon for a user to select to get readings from the ECG. Upon touching or activating a symbol, the system may trigger an action to begin utilizing a specific component of the health monitoring system to capture electronic signals utilizing for reading data. For example, the temperature sensor, blood pressure sensor, heart rate sensor, glucose sensor, ECG sensor, audio sensor, or camera may be utilized to capture measurements form the user.

A blood sugar screen 405 may illustrate a symbol or icon as related to blood sugar/glucose readings. Upon selection of the icon via a touch input or other interface input (e.g., voice, stylus, button or other control device, etc.), the system may conduct a reading of the users blood sugar.

A dietary screen 407 may inquire questions about a user's food intake. The system may prompt a question that can be answered via a voice input (e.g. through a mic) or via a keyboard or another system. The food intake may be utilized for blood sugar level for monitoring glucose. The system may then utilize the food input to monitor certain readings or adjust thresholds. The system may allow for a meal tag to be introduced to the reading and thus modify any data.

An image screen 409 may be utilized to take a picture of the user. In another scenario, the image may be utilized to identify skin conditions or other issues that may be viewed via a picture. For example, a skin color may be identified utilizing the image. In another example, a rash may be identified.

At ECG reading screen 411, the system may show various metrics utilized to read the ECG. The ECG reading may include the type of wave, and amplitude. The readings may be in microvoltage (mV) or another type of unit.

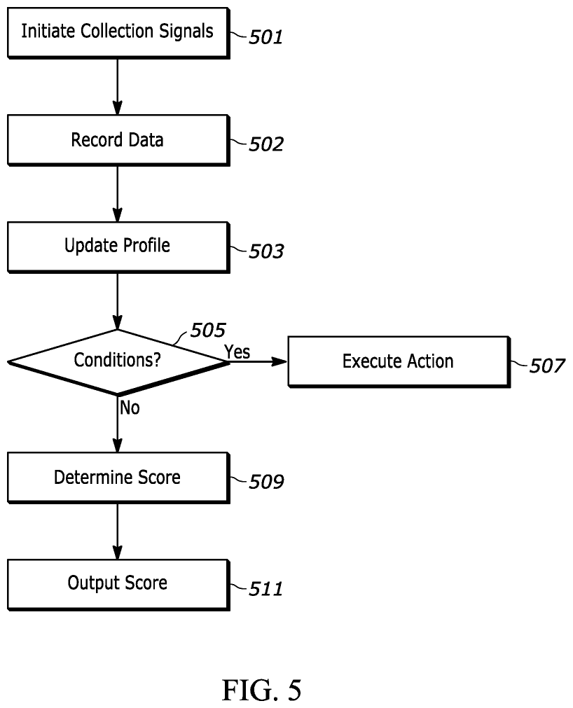

FIG. 5 illustrates a flow chart related to the health monitoring system. At step 501, the system may receive an initiation to collect signals from one or more sensors. The initiation may result in a user interface screen activating an icon for one or more of the sensors. The input may activate a switching topology to initialize the various sensors as applicable to the interface. For example, a switch associated with the blood pressure monitor may be activated if a user chooses to utilize the blood pressure monitor.

The monitoring device may also include at least one circuit electronically coupled to or integrated with the at least one switch. The at least one circuit may further be operably connected to the switch control unit of the monitoring device. Each circuit may include an analog to digital converter (ADC), a digital signal processor (DSP), and a signal storage and transmitter (SST). In some embodiments, one circuit may be electronically coupled to or integrated with one switch. In some other embodiments, one circuit may be electronically coupled to or integrated with more than one switch. The monitoring device 100 may include a processor 101 to execute any flow charts, algorithms, and/or processes.

In response to activation of the switch, the gateway controller may download or utilize a driver to provide an application program interface to the one or more sensors utilize to capture data. The driver may be utilized to manage interfaces of the sensor that are linked to hardware or other low-level services of the sensors. In one example, the personal health monitoring system may be in communication with the remote server to update a driver associated with one or more sensors. The API may be utilized in conjunction with the driver to allow the gateway controller to communicate with the one or more various sensors, including varying sensors from different manufactures.

At step 502, the system may collect and record the data utilizing the sensors. Thus, it may be assumed that the API of the gateway controller or PHMS has allowed for communication and operation of the one or more sensors (e.g., camera, blood pressure monitor, ECG, etc.). The sensor may communicate such readings to the gateway controller, which may store the readings in memory or communicate the data remotely, or a hybrid-approach. The system may determine the appropriate switch to activate and the appropriate driver or API to execute in response to the input received at the GUI.

At step 503, the system may update the user profile based on the data. For example, the historic data of such readings may be utilized and compared. The readings may be stored with respect to the user. Furthermore, various thresholds may be updated based on the analytics engine or model that is utilized for a particular user.

At step 505, the system may determine if any conditions exist, including any emergency conditions. The signals may be analyzed and to the extent they exist, an action may be executed at 507. Any health conditions or emergency conditions may be notified based on a single reading of a single sensor or combining multiple readings from multiple sensors. For example, a certain response may exists if both the ECG data and blood pressure data indicate a possible health condition. In yet another example, the pattern recognition algorithm may be configured to identify abnormal health condition patterns and/or symptoms based on the received data. These abnormal health condition patterns and/or symptoms may be taken by the analytics engine as indications of potential health issues or concerns of the user. Thereupon, the remote server may generate a health alert and may then transmit the health alert to the monitoring device. In urgent conditions, the system may call an emergency contact (ICE) associated with the user profile or an emergency operator based on the reading. If no emergency conditions exist, the system may run the data and readings through the analytics engine to determine a health assessment score at step 509. The health index or score may be determined as discussed above.

At step 511, the system may output a health score utilizing the analytics engine. The analytics engine may be located on-board, at a remote server, or be based on a hybrid approach. The remote server may utilize the analytics engine configured to analyze the received data to ultimately determine the user's health conditions. The received data may include, but not limited to, data related to the user's health measurement events and the user's health profiles. The analytics engine may include one or more algorithms configured to monitor, identify, and/or predict a health issue or concern based on the received data. The thresholds for identifying any health issues or concerns may be preset by health experts. The thresholds may also be based on the data stored in the health-related database of the data factory. In addition, the thresholds may be determined by a pattern recognition algorithm implemented in the analytics engine.

The model or engine may be utilized to factor one or more of the readings to determine an overall health. For example, if a user's record for body temperature is extreme (e.g., more than 104 degrees) the system may output an alert and notification to address a health condition via the user interface or to a remote system. In yet another embodiment, if the body temperature is slightly high but combined with another off-target recording from one of the sensors (e.g., high blood sugar), the system may trigger an alert that may not occur if only of the readings are off. Thus, the system may utilize varying thresholds that are combined with the other readings and a user's profile to determine the health report. Historic data and variance in the ranges from the sensor reading will be utilized to identify health conditions from the analytics engine or machine learning model, according to one embodiment.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A health monitoring system utilized for a user, comprising:
an enclosure configured to hold one or more components of the health monitoring system, the enclosure including:
a wireless transceiver configured to communicate health data to a remote server;
a camera configured to capture an image associated with a user;
a temperature sensor configured to capture temperature data indicative of a body temperature indicative of the user;
a blood pressure sensor configured to capture blood pressure data indicative of a blood pressure of the user;
a heart rate sensor configured to capture heart rate data indicative of a heart rate of the user;
a glucose sensor configured to capture glucose data indicative of a glucose level of the user;
an electrocardiogram (ECG) configured to record an electrical signal associated with a heart of a user to capture ECG data;
a processor in communication with the wireless transceiver, camera, temperature sensor, blood pressure sensor, heart rate sensor, glucose sensor, and ECG, wherein the processor is further programmed to:
receive the images, the temperature data, the blood pressure data, the heart rate data, and the glucose data, the ECG data;
send, via the wireless transceiver, to the remote server the images, the temperature data, the blood pressure data, the heart rate data, and the glucose data, the ECG data;
utilizing a machine learning model and the image, the temperature data, the blood pressure data, the heart rate data, and the glucose data, the ECG data , identify one or more health conditions associated with the user; and
in response to the identifying the one or more health conditions exceeding a health threshold, output a health report indicative of one or more health assessments indicating a healthy condition or an illness associated with the user.

2. The health monitoring system of claim 1, wherein the processor is further configured to receive login information associated with the user from a human machine input interface; and
associate one or more of the temperature data, the blood pressure data, the heart rate data, and the glucose data, the ECG data with the user utilizing the login information.

3. The health monitoring system of claim 1, wherein the processor is configured to utilize the image and one or more of the temperature data, the blood pressure data, the heart rate data, and the glucose data, the ECG data to identify a skin condition associated with the user.

4. The health monitoring system of claim 1, wherein the processor is further programmed to send, in response to exceeding the health threshold, the health report to one or more associated tags.

5. The health monitoring system of claim 1, wherein the health report outputs an index or a numerical value representing a health profile of the user.

6. The health monitoring system of claim 1, wherein the processor is further programmed to utilize pattern recognition algorithm to identify abnormal health condition patterns or symptoms based on the received data.

7. The health monitoring system of claim 1, wherein the health monitoring system further includes a display in communication with the processor, the display configured to output a graphical user interface associated with the health monitoring system.

8. The health monitoring system of claim 1, wherein the machine learning model is located at the remote server.

9. The health monitoring system of claim 1, wherein the processor is further programmed to, in response to the identifying the one or more health conditions exceeding a health threshold, output a health report indicative of one or more health assessments indicating a healthy condition or an illness associated with the user.

10. The health monitoring system of claim 1, wherein the processor is further programmed to, in response to utilizing a machine learning model and the image, the temperature data, the blood pressure data, the heart rate data, and the glucose data, the ECG data, output a health score indicative of one or more health assessments associated with the user.

11. A health monitoring system utilized for a user, comprising:
a wireless transceiver configured to communicate health data to a remote server;
one or more processors in communication with the wireless transceiver wherein the one or more processors are collectively further programmed to:
upon receiving an input at a user interface of the health monitoring system, activate a switch to activate one or more sensors in communication with the health monitoring system, wherein the one or more sensors are configured to collect one or more of an image of the user, temperature data, blood pressure data, heart rate data, glucose data, or echocardiogram data;
send, via the wireless transceiver, to the remote server the images, the temperature data, the blood pressure data, the heart rate data, and the glucose data, the ECG data;
utilizing a machine learning model and one or more of the image, the temperature data, the blood pressure data, the heart rate data, and the glucose data, the ECG data, identify one or more health conditions associated with the user; and
in response to the identifying the one or more health conditions exceeding a health threshold, output a health report indicative of one or more health assessments indicating a healthy condition or an illness associated with the user.

12. The system of claim 11, wherein the one or more processors is a single processor located remote from the health monitoring system.

13. The system of claim 11, wherein the one or more processors includes a single processer located at the health monitoring system and one or more remote processors located remote from the health monitoring system.

14. The system of claim 11, wherein the switch that is activated is associated with the input at the user interface.

15. The system of claim 11, wherein the processor is further configured to initiate a driver in response to the input at the user interface, wherein the driver is configured to control and manage hardware of the one or more sensors in communication with the health monitoring system.

16. The system of claim 11, wherein the one or more sensors are in communication with the health monitoring system via a wireless communication protocol.

17. A method of monitoring a health of a user utilizing a personal health monitoring system, wherein the method includes:
utilizing a camera, receiving an image associated with a user;
utilizing a temperature sensor, receiving temperature data indicative of a body temperature indicative of the user;
utilizing a blood pressure monitor, receiving blood pressure data indicative of a blood pressure of the user utilizing a blood pressure monitoring;
utilizing a heart rate sensor, receiving heart rate data indicative of a heart rate of the user;
utilizing a glucose monitor, receiving glucose data indicative of a glucose level of the user;
utilizing an electrocardiogram (ECG), receiving an electrical signal associated with a heart of a user to capture ECG data;
receiving, via one or more processor, the images, the temperature data, the blood pressure data, the heart rate data, and the glucose data, the ECG data;
sending, via the wireless transceiver, to the remote server the images, the temperature data, the blood pressure data, the heart rate data, and the glucose data, the ECG data;
utilizing a machine learning model and the image, the temperature data, the blood pressure data, the heart rate data, and the glucose data, the ECG data, identifying one or more health conditions associated with the user; and
in response to the identifying the one or more health conditions exceeding a health threshold, outputting a health report indicative of one or more health assessments indicating a healthy condition or an illness associated with the user.

18. The method of claim 17, wherein the one or more processors is in communication with a wireless transceiver, camera, temperature sensor, blood pressure sensor, heart rate sensor, glucose sensor, and ECG, and the one or more processors are collectively further programmed to execute two or more steps.

19. The method of claim 17, wherein the health threshold is dynamic in response to the machine learning model.

20. The method of claim 17, wherein the method includes outputting, to a display of the health monitoring system, one or more of the health report or health conditions.
